(19) United States
(12) Patent Application Publication
(10) Pub. No.: US 2019/0022301 A1
(43) Pub. Date: Jan. 24, 2019

(54) Enema and Vaginal Cleaning Apparatus

(71) Applicant: Matthew Fields, Covina, CA (US)

(72) Inventor: Matthew Fields, Covina, CA (US)

(21) Appl. No.: 16/039,271

(22) Filed: Jul. 18, 2018

Related U.S. Application Data
(60) Provisional application No. 62/534,112, filed on Jul. 18, 2017.

(51) Int. Cl. A61M 3/02; A61M 39/28

(52) U.S. Cl. CPC A61M 3/0245; A61M 3/0279; A61M 2210/1475; A61M 2210/1064; A61M 39/28

(57) ABSTRACT

An apparatus for holding and administering an enema and vaginal cleaning includes a container with an opening and a port. A fitting is provided having a threaded portion, and a neck portion. A nut is configured to engage the threaded portion, with a first seal seated between the flange portion and the container, and a second seal seated between the nut and the container. A hose has a first end and a second end, with the first end coupled to the fitting adjacent the neck portion, and a tip has an insertion end for inserting into the hose. The tip is configured for emitting the fluid from the hose, as the liquid moves from the container, through the hose, and out of the tip under gravity.

### RELATED APPLICATIONS

This application claims the benefit of the priority filing date of U.S. provisional patent application Ser. No. 62/534,112, entitled “Enema and Vaginal Cleaning Apparatus,” which was filed on 18 Jul. 2017, and which is incorporated herein by reference in its entirety.

### BACKGROUND

The present apparatus generally relates to fluid transfer devices. More specifically, the present invention relates to an enema and vaginal cleansing apparatus.

Numerous enema and similar evacuation apparatus exist in the art. For example, U.S. Pat. No. 1,747,391 issued to Sarason (patented Feb. 18, 1930) teaches an enema apparatus comprising an injection tube, a yieldable water bag, and a rubber hose. The rubber hose connects the injection tube with the water bag. The water bag has a filling opening and a closing screw, and is adapted to force the water into the injection tube under pressure exerted with the hands. A rubber ball is slidable on the injection tube and is adapted to be clamped between the thighs of the user and to hold the injection tube so that the user's hands are free for exerting pressure on the water bag.

Similarly, U.S. Pat. No. 2,690,181 issued to Boyer (pub. Sep. 28, 1954) teaches an enema applicator comprising a unitary body of plastic material adapted for storing, transporting and introducing an enema solution in predetermined, measured quantities. The body has a hollow, collapsible container for the solution and is provided with an opening in the wall thereof. There is also a hollow, elongated nozzle in communication with the opening and extends outwardly from the container. The walls of the nozzle are substantially thicker than the walls of the container to maintain the nozzle relatively rigid while manipulating the nozzle during the filling and expelling operations. The nozzle has a tubular portion of uniform diameter throughout its length at the free end of the nozzle. A cylindrical neck portion of relatively greater diameter is disposed at the zone of juncture between the nozzle and the container, and a hollow, intermediate portion of conical form between the tubular portion and the cylindrical portion. The walls of the nozzle progressively increases in thickness from the container to the tubular portion.

There are various problems inherent in these enema devices and other related apparatus that are currently known in the art. Among others, several examples of these shortcomings include: (a) high purity composition characteristics combined with airtight storage features that enables sanitary preservation for heat/air sensitive solutions to be used at a later time; (b) interchangeable parts that can be repaired or replaced to avoid having to purchase a whole new apparatus, and which allows for disassembly so that the apparatus can be thoroughly cleaned; and (c) vaginal hygiene components, so that the device can double as a vaginal cleansing apparatus.

The present apparatus addresses these shortcomings and presents several advantages over the prior art. When the proper tip is applied to the enema delivery tube of the present apparatus, it serves the purpose of introducing a solution into a female's vagina for retention, similar to an enema, for the purpose of vaginal cleansing and toxin removal, usually used in various bodily detoxification programs.

With regard to additional advantages, the present invention has a threaded pattern around the top orifice that allows a variety of lids/rings to be tightened atop the reservoir rendering this apparatus an airtight storage container for whatever solution it may contain, and can be placed in the refrigerator if necessary. Furthermore specialty lids acting as a debris shield can allow airflow for the proper gravity feed of liquid while keeping foreign objects and debris from entering the reservoir and coming into contact with the solution. The invention also features a high purity glass container and heavy-duty chemically inert components.

Regarding yet other advantages, having interchangeable parts allows present invention to be used long term, and can be upgraded or repaired using replacement parts. The enema device need not be discarded after several uses because each component can be disassembled and cleaned.

The liquid containing reservoir can be a variety of sizes to provide a smaller and easier to handle apparatus and/or to accommodate a smaller quantity of liquid depending on the purpose, type of liquid solution and/or the user's physiological makeup. The reservoir also features a flat bottom, allowing it to be placed to be placed on an elevated horizontal surface instead of being suspended from the attached hanger. The top reservoir entrance should be threaded to allow many purposeful covers to be applied and fixed in place atop the reservoir, for example, a sealed top for an airtight storage container or a mesh cover to act as a debris shield in order to keep contents uncontaminated.

### SUMMARY

An apparatus is disclosed for holding and administering a liquid for an enema and alternatively for vaginal cleaning. The apparatus includes a container configured for holding the liquid. The container has both an opening for receiving the liquid and a port forming a bore through the container preferably at the bottom of the container. A fitting is provided having a threaded portion, and a neck portion, with a flange portion separating the threaded portion and the neck portion. A nut, configured to engage the threaded portion, has a diameter larger than the port, with a first seal seated between the flange portion and the container, and a second seal seated between the nut and the container.

A hose is provided having a first end and a second end. The first end of the hose is coupled to the fitting adjacent the neck portion, and the hose is preferably made of a flexible material. A tip is also provided, the tip having an insertion end for inserting into the second end of the hose, and a stopper flange for regulating a distance the tip can be inserted into the second end of the hose. The tip is configured for emitting the fluid from the hose, as the liquid moves from the container, through the hose, and out of the tip under gravity.

In various alternative embodiments, the container may include a re-sealable closure, and may be made of a jar having a threaded lid portion. The container is preferably configured such that the opening is oriented above the port, and the threaded portion is preferably sized for insertion through the port, with the flange portion being larger in diameter than the port. A tightening section is preferably provided adjacent the flange portion for manipulating the fitting and tightening it against the container.

In yet other alternative embodiments, the neck portion may comprise a hose anchor, and the first seal and the second seal each may comprise o-ring type sealing rings. Preferably the hose is made of a chemically inert flexible material, and the apparatus includes a hose clamp for closing off fluid flow through the hose. The hose clamp is preferably a pressure clamp adapted to slide along the hose, and the tip comprises a specialized tip having multiple liquid ports oriented in different directions.

The apparatus may also be characterized as an enema and vaginal cleaning apparatus including a non-flexible vessel comprising a material chosen from the list of glass or ceramic. The vessel includes a port configured to accept a fitting, with the fitting comprising a high purity plastic and the fitting sealed to the vessel with two high purity rubber sealing rings. A threaded nut is provided to adapt the vessel to a chemically inert flexible delivery tube, and the delivery tube placed in communication with a rectal tip adapted for introduction of a liquid in the vessel into the rectum of a user. A clamp is coupled to the delivery tube between the vessel and the rectal tip, and the apparatus is provided with a closure and a removable hanger that allows the user to suspend the vessel in an elevated position above the rectal tip to achieve a gravity driven flow.

### BRIEF DESCRIPTION OF THE FIGURES

FIG. 1 illustrates a perspective exploded view of the enema and vaginal cleaning apparatus.

FIG. 2 illustrates a perspective view of a first tip and a second tip for fixing to the enema and vaginal cleaning apparatus.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

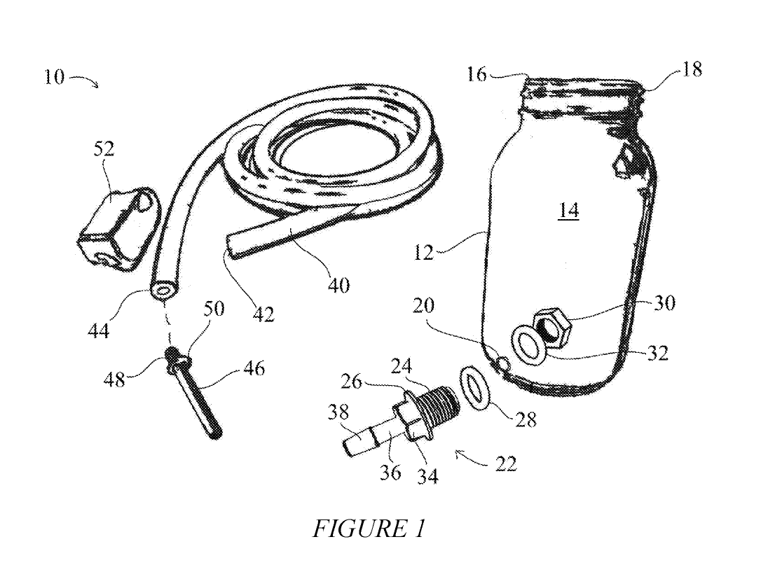

Referring to FIG. 1, an enema and vaginal cleaning apparatus 10 generally comprises a container 12, which can vary in size, and which is useful for storage, measuring, and administration of a liquid composition (not shown), for example, a saline enema. The container 12 is preferably made of a non-flexible glass or ceramic material, and comprises a reservoir portion 14 and an opening 16 for accepting ingress of the liquid composition. The opening preferably comprises threading 18 or another mechanism for sealing a lid (not shown) on the container 12.

The container 12 also comprises a port 20, preferably located toward a bottom end of the container 12 for egress of the liquid composition. The port 20 is preferably a bore formed through the non-flexible glass or ceramic material. A fitting 22 is provided, having a threaded portion 24 for inserting through the port 20, and a flange portion 26 for abutting a first rubber sealing ring 28, which abuts the outside of the container 12. The threaded portion 24 engages a threaded nut 30, which abuts a second sealing ring 32 abutting the inside of the container 12. A tightening portion 34 adjacent the flange portion 26 opposite the threaded portion 24 allows a user to hold the fitting static as the threaded nut 30 is tightened onto the threaded portion 24. In this manner, the fitting 22 is sealed relative to the container 12, and liquid entering the container 12 through the opening 16 exits the container through the fitting 22.

Opposite the threaded portion 24, the fitting 22 comprises a neck 36 and a hose anchor 38 for retaining a hose 40 on the fitting 22. The hose 40 is preferably a flexible walled enema delivery tube which is chemically inert to the contents of the container and preferably hypo-allergenic, comprising a material appropriate for insertion into a user's body. Although shown in a coiled configuration as illustrated, the hose 40 is preferably of sufficient length to be easily manipulated by a user, while the container 12 remains at a sufficient height to allow the liquid to exit the container through the fitting 22 and through the hose 40 under gravity. A first end 42 of the hose 40 is preferably attached to the hose anchor 38, while a second end 44 of the hose 40 is attached to a tip 46 for insertion into the user's rectum.

The tip 46 preferably includes an insertion end 48 for inserting into the hose under sufficient tension to hold it therein, and a stopper flange 50 for regulating the distance the tip 46 can be inserted into the hose 40. A hose clamp 52 is preferably provided for closing off the hose 40, and thus arresting the flow of the liquid from the container 12. In the illustrated embodiment, the hose clamp 52 is a pressure clamp that slides over the hose 40, and thus can engage the hose 40 at any point along its length according to preference.

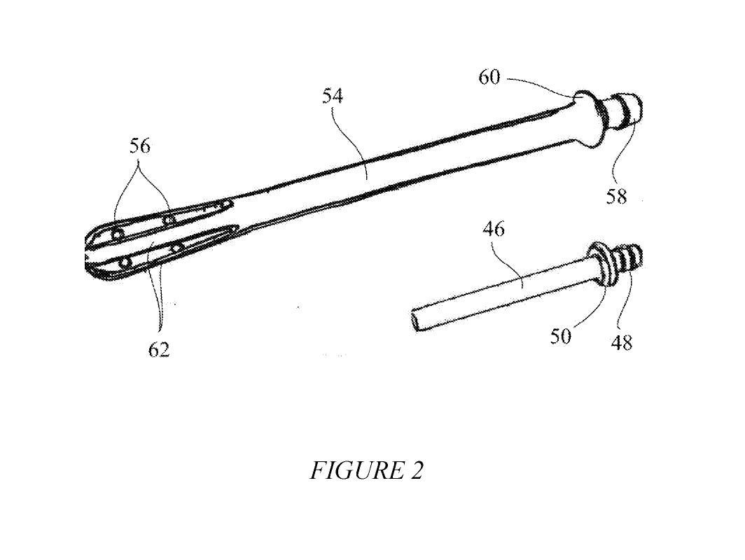

Referring to FIG. 2, the tip 46 is shown alongside a specialized tip 54, which is elongated and has multiple liquid ports 56 for enhanced irrigation. In the illustrated embodiment, the specialized tip 54 includes an alternative insertion end 58 for connection to the hose 40 via insertion and an alternative stopper flange 60 for regulating the distance the specialized tip 54 can be inserted into the hose 40. Also shown in the illustrated view, the liquid ports 56 are arranged in rows, and are separated by a series of fins 62 to direct the flow of liquid from the specialized tip 54 in all directions.

In order to use the enema and vaginal cleaning apparatus 10, the tip 46 (or, alternatively, the specialized tip 54) is inserted into the hose 40, with the hose clamp installed in a preferred position along the hose 40. An end of the hose 40 opposite from the tip 46 the hose anchor 38 is inserted into the hose 40 such that the hose is installed on the neck 36 of the fitting 22. Before or after connecting the hose 40 to the fitting 22, the threaded portion 24 is inserted through the container 12 and engaged by the threaded nut 30, with the first rubber sealing ring 28 and the second rubber sealing ring 32 sealing the port 20. Preferably the opening 16 is provided with a closure and a removable hanger (not shown) that allows the user to suspend the container 12 in an elevated position above the tip 46 to achieve a gravity driven flow of the liquid. In various alternative embodiments, the device is formed of materials having a long service life and which can be repaired and/or upgraded/customized for the user's personal preference. For example, a larger delivery tube having a larger circumference can be used for increased flow, and a quick-disconnect style fitting 22, and other alternatives can be used for easier cleaning, etc.

With the components of the enema and vaginal cleaning apparatus 10 assembled, the container 12 may be filled with the liquid, the opening covered 16, and the container 12 brought to an elevated position with the hose clamp 52 closing off liquid flow through the hose 40. The tip 46 can then be inserted into a user's rectum or vagina, and the hose clamp 52 opened to allow the liquid to flow under gravity through the fitting 22, hose 40 and tip 46 (or, alternatively, the specialized tip 54) and into the user.

When introducing any type of enema or vaginal cleansing/detoxification solution into the body, it is of utmost importance to make sure that the solution to be administered is free of any contaminants. Conventional enema apparatuses are produced from many materials, for example, Latex rubber, silicone rubber, various plastic and metal compounds. These reservoirs pose a risk factor of solution contamination due to the leaching nature of these material compositions which, in turn, poses a risk to the users of such devices. To eliminate associated risks, the reservoir and all parts that come into direct solution contact are preferably of a chemically inert material. Glass and/or ceramic, which is known to be inert in such situations is the composition of the container 12. To ensure a high purity device, any plastic or rubber compounds particularly used for this apparatus are to be compliant with any one or more of the purity testing standards/certifications set forth by such establishing national/international certification entities, for example the USP (United States Pharmacopeia), FDA (Food and Drug Administration), NSF International (Nation Sanitation Foundation), among others.

The main function of this apparatus, is intended for administering fluid into the body via entrance through a sphincteric orifice of the body. Based on the fact that the vagina and anus are both entrances into the body consisting of a sphincteric orifice leading to a canal, this apparatus's primary intentional use would be equally for the purpose/process of vaginal douching or vaginal retention enema as well as rectal enema. The specialized tip 54, may be used for vaginal solution administration since it has a greater length dimension than the rectal tip 46 and consists of a larger end shape with multiple liquid ports 56, for the fluid to exit, as opposed to a single exit opening as shown in tip 46, in order to achieve a multiple angular dispensing of the fluid.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. An apparatus for holding and administering a liquid for an enema and for vaginal cleaning, the apparatus comprising:
a container configured for holding the liquid, the container having an opening for receiving the liquid and a port forming a bore through the container;
a fitting having a threaded portion, and a neck portion, with a flange portion separating the threaded portion and the neck portion;
a nut configured to engage the threaded portion, the nut having a diameter larger than the port;
a first seal seated between the flange portion and the container, and a second seal seated between the nut and the container;
a hose having a first end and a second end, with the first end coupled to the fitting adjacent the neck portion, the hose comprising a flexible material;
a tip having an insertion end for inserting into the second end of the hose, a stopper flange for regulating a distance the tip can be inserted into the second end, the tip configured for emitting the fluid from the hose; and
wherein the liquid moves from the container, through the hose, and out of the tip under gravity.

2. The apparatus of claim 1 wherein the container comprises a re-sealable closure.

3. the apparatus of claim 1 wherein the container comprises a jar having a threaded lid portion.

4. The apparatus of claim 1 wherein the container is configured such that the opening is oriented above the port.

5. The apparatus of claim 1 wherein the threaded portion is sized for insertion through the port, and the flange portion is larger in diameter than the port.

6. The apparatus of claim 1 further comprising a tightening section adjacent the flange portion.

7. The apparatus of claim 1 wherein the neck portion comprises a hose anchor.

8. The apparatus of claim 1 wherein the first seal and the second seal each comprise o-ring type sealing rings.

9. The apparatus of claim 1 wherein the hose comprises a chemically inert flexible material.

10. The apparatus of claim 1 further comprising a hose clamp for closing off fluid flow through the hose.

11. The apparatus of claim 10 wherein the hose clamp is a pressure clamp adapted to slide along the hose.

12. The apparatus of claim 1 wherein the tip comprises a specialized tip having multiple liquid ports oriented in different directions.

13. An Enema and Vaginal Cleaning Apparatus comprising:
a non-flexible vessel comprising a material chosen from the list of glass or ceramic;
the vessel comprising a port configured to accept a fitting;
the fitting comprising a high purity plastic;
the fitting sealed to the vessel with two high purity rubber sealing rings;
a threaded nut configured to adapt the vessel to a chemically inert flexible delivery tube;
the delivery tube placed in communication with a rectal tip adapted for introduction of a liquid in the vessel into the rectum of a user;
a clamp coupled to the delivery tube between the vessel and the rectal tip;
and wherein the apparatus is provided with a closure and a removable hanger that allows the user to suspend the vessel in an elevated position above the rectal tip to achieve a gravity driven flow.
